(19) United States
(12) Patent Application Publication
(10) Pub. No.: US 2002/0022639 A1
(43) Pub. Date: Feb. 21, 2002

(54) Packaging regimen of pseudoephedrine hydrochloride and fexofenadine hydrochloride

(76) Inventors: Douglas E. Randall, Belle Meade, NJ (US); James M. Nicholas, Overland Park, KS (US)

Correspondence Address: AVENTIS PHARMACEUTICALS, INC., PATENTS DEPARTMENT, ROUTE 202-206, P.O. BOX 6800, BRIDGEWATER, NJ, 08807-0800, US

(21) Appl. No.: 09/848,463

(22) Filed: May 3, 2001

Related U.S. Application Data
(60) Provisional application No. 60/202,323, filed on May 5, 2000.

(30) Foreign Application Priority Data
Dec. 18, 2000 (GB) 0030802.3

(51) Int. Cl. A61K 31/445; A61K 31/135; B65D 85/42

(52) U.S. Cl. 514/317; 514/653; 206/538

(57) ABSTRACT

A package for dispensing two or more pharmaceutically active compounds is described and claimed. In one of the embodiments of this invention, the package dispenses essentially: a) a container 1 to dispense drug A having therapeutically effective amounts of fexofenadine or its pharmaceutically acceptable addition salt; and b) a container 2 to dispense drug B containing therapeutically effective amounts of a combination of fexofenadine and pseudoephedrine or their pharmaceutically effective addition salts. In this embodiment there is also provided an indicia to distinguish between the drugs A and B in the containers 1 and 2. Various preferred embodiments of the package of this invention are also described and claimed.

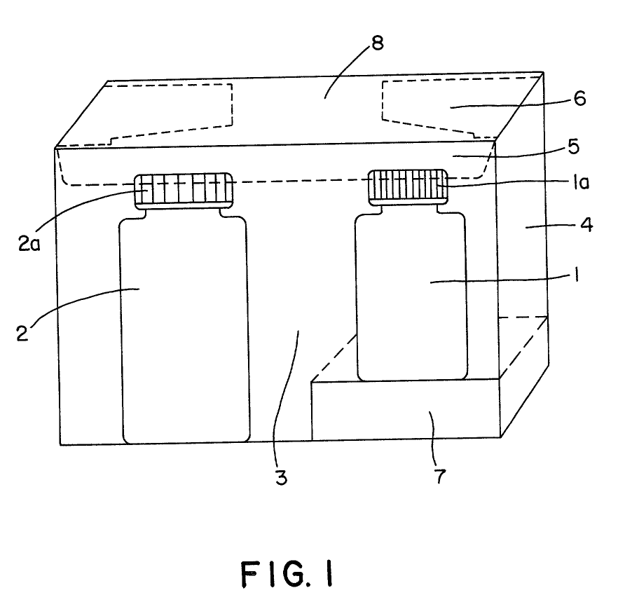

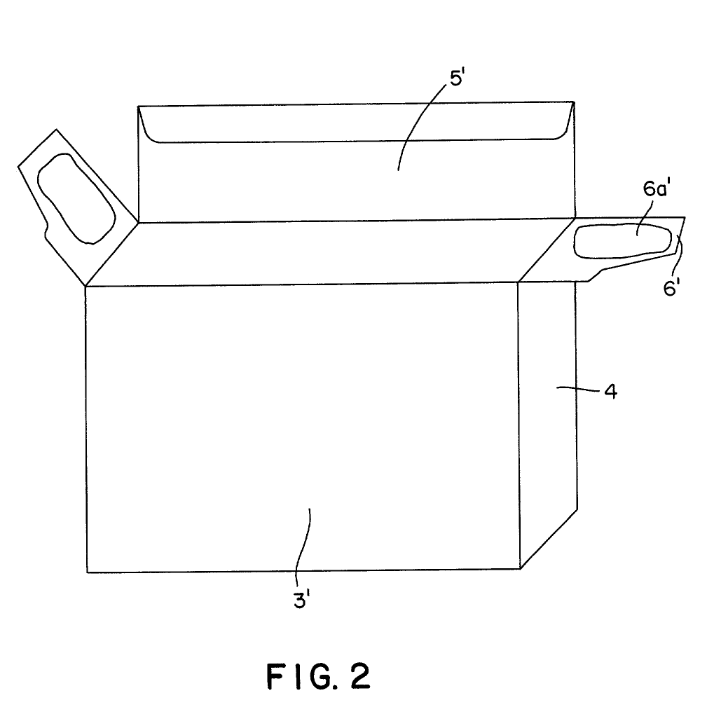

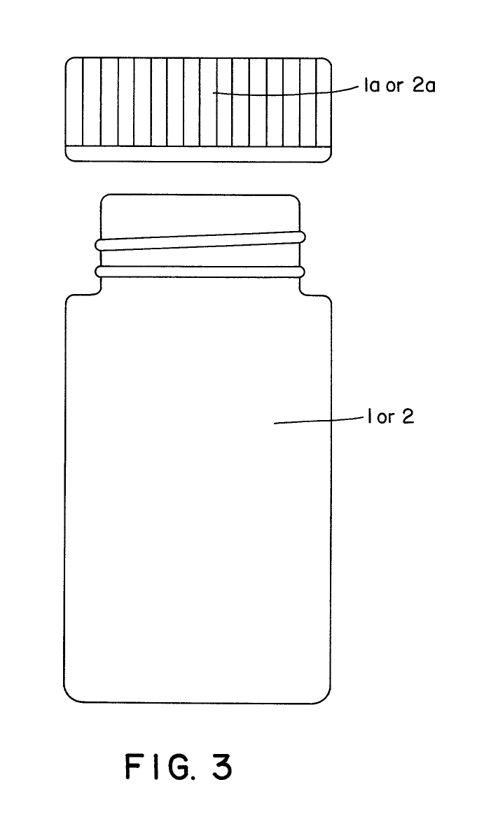

This application claims the benefit of U.S. Provisional Application No. 60/202,323, filed May 5, 2000.

### BACKGROUND OF THE INVENTION

1. Field of the Invention

The present invention relates to a mode of packaging of two separate drugs, via two separate dosage units, which proves useful from a convenience perspective. More specifically, this application details the packaging of two drugs which contain fexofenadine hydrochloride and pseudoephedrine hydrochloride. The dosage unit containing pseudoephedrine hydrochloride is to be administered during the daytime and the dosage unit that is void of pseudoephedrine hydrochloride is to be administered during the nighttime.

2. Description of the Prior Art

Modes of packaging two separate drugs together as a daytime and nighttime packaging scheme have been established in the art. A few examples include:

E. Knudsen describes in U.S. Pat. No. 4,295,567, a packaging regimen in the form of a blister pack which dispenses two separate dosage units that treat respiratory disorders.

Weinstein, et al. disclose in International Application No. WO 99/21556, published on May 6, 1999, a regimen to treat rhinitis which contains two different medication dosage units and that may incorporate bottles, blister packages or pouches.

However, the current art is void of a single packaging regimen that includes fexofenadine hydrochloride and pseudoephedrine hydrochloride in particular. In addition, the prior art does not provide for the two separate drugs to be dispensed specifically as containers or bottles which are within a small convenient uni-package that is a box. Furthermore, the instant invention provides for the advantage of the display of a prescription card once and a single copayment for both fexofenadine hydrochloride and pseudoephedrine hydrochloride, and each being contained in two separate dosage units. Under the current state of the art, two separate copayments would be required to receive these two therapeutic drugs. More importantly, under current practice it is not possible for the prescribing physician to write a single prescription involving both of these types of drugs.

Accordingly, it is the purpose of this invention to provide a single package as a box within which contains two separate drugs. More specifically, it is the aim to provide a single package as a box that contains i) a drug A which is comprised of fexofenadine hydrochloride and ii) a drug B which is comprised of fexofenadine hydrochloride and pseudoephedrine hydrochloride. Beyond the current state of the art, it is the aim of the present invention to provide features from a convenience perspective in that the consumer need only present a prescription card once to receive both fexofenadine hydrochloride and pseudoephedrine hydrochloride to treat their condition. This feature promotes the ability to dispense these two drugs together, combined under one single copayment, as opposed to two copayments.

### SUMMARY OF THE INVENTION

The present invention combines the single packaging aspect in the form of a box to dispense therapeutically effective amounts of both fexofenadine hydrochloride and pseudoephedrine hydrochloride coupled with the advantage of the consumer paying a single payment and the single presentation of a prescription card.

The present invention provides for a package to dispense two or more pharmaceutically active compounds which contain: (a) a container 1 to dispense drug A that has a therapeutically effective amount of fexofenadine or a pharmaceutically acceptable addition salt and (b) a container 2 to dispense drug B which has a therapeutically effective amount of fexofenadine and pseudoephedrine or their pharmaceutically acceptable addition salts; where an indicia is provided to distinguish between the drugs A and B and the containers 1 and 2.

### BRIEF DESCRIPTION OF THE DRAWINGS

FIG. I is a drawing of the single package as a box with the front portion of the box cut out to show how the containers are positioned while inside the package.

FIG. II is a drawing of the single package as a box with the flap of the box open to show the point at which to insert the containers into the box.

FIG. III is a drawing that shows the container as an open bottle where the screw top cap is positioned directly above the opening.

### DETAILED DESCRIPTION OF THE INVENTION

It has been discovered that a consumer is now able to present a prescription card once and pay a single payment, yet receive two separate drugs.

As used herein, the package is meant to be any of the means by which drugs may be dispensed in one unit. For example, but not limited to, packaging types may include different geometric configurations of boxes. Such examples of geometrical configurations include rectangular, circular, square or cylindrical boxes. The preferred method of packaging for the present invention is as a rectangular box. It is a further preference that the present invention be in the form of a convenient, single package or uni-package. Convenient is meant to apply in reference to the consumer upon receiving the prescription or over the counter medication as one unit and paying one single payment and/or presenting their prescription card, if needed, only once.

As used herein the container is meant to include any suitable container for housing drugs A and B. Containers within the present invention are not limited to medicinal bottles. Other examples may include canisters, blister packs, tubes or individual packets. Medicinal may refer to any medications or medicaments in the form of capsules, caplets, tablets or liquid formulations. The medications may be administered either through prescription or as over-the-counter medications.

A therapeutically effective amount of the compound refers to an amount sufficient to create the desired effect. Therapeutically effective amounts of the compounds of the present invention can be administered to a subject by any one of the acceptable methods. For example, this may include the oral administration of capsules, caplets, tablets or liquid formulations. The term “therapeutically effective amount” does not necessarily mean that there is a complete cure of the condition. Many factors are considered when determining the therapeutically effective amount. Some examples of these factors include but are not limited to: the specific condition involved; the degree or intensity of the condition; the response by the individual subject; which compound is being administered; the mode in which it is administered; and the species of mammal; and its size, age and overall general health.

As used herein, the term “subject” is meant to refer to any warm blooded animal. More specifically, it refers to a mammal which has a condition that is treatable by different dosage units that contain fexofenadine hydrochloride and pseudoephedrine hydrochloride. Further examples of such animals may include but are not limited to guinea pigs, dogs, cats, rats, mice, horses, cattle, sheep and most preferably, humans.

Indicia herein refers to a distinguishing feature of the two containers within the package. Such examples may include a feature such as size, color, shape, or a marking so as to indicate which container contains drug A and which contains drug B. The preferred indicia of the present invention is size.

The present invention allows the consumer to purchase a convenient, single package or uni-package in the form of a box that dispenses two or more pharmaceutically active compounds which contain: (a) a container 1 to dispense drug A that has a therapeutically effective amount of fexofenadine or a pharmaceutically acceptable addition salt thereof and (b) a container 2 to dispense drug B which has a therapeutically effective amount of fexofenadine and pseudoephedrine or their pharmaceutically acceptable addition salts; where an indicia is provided to distinguish between the drugs A and B and the containers 1 and 2. As used herein, the term pharmaceutically active compounds is meant to refer to drugs that could potentially be useful in the prevention, diagnosis, and treatment of human disease. (Goodman & Gilman's, The Pharmacological Basis of Therapeutics, 9^{th} Edition, page 1, lines 8-9, McGraw Hill, 1996).

In one aspect of this invention, the package in accordance with this invention includes size and color as indicia. In a further preferred embodiment, the package according to this invention has the size of the container as the indicia. More specifically, drug A is contained in the smaller bottle and drug B is contained in the larger bottle and both of which are contained within the convenient uni-package as a box. In one aspect of this invention, the package in accordance with this invention has containers 1 and 2 in the form of medicinal bottles. The container referred to herein can be made in many different ways. Representative examples include, but are not limited to descriptions provided in U.S. Pat. No. 4,3691,382 and U.S. Pat. 5,850,940 which are herein incorporated by reference. Drug A is indicated for nighttime use and drug B for daytime use, both of which comprise pharmaceutical carriers and formulating aids.

The forms of drugs A and B may include capsules, caplets, tablets and liquid formulations. As a preferred embodiment of the invention, drugs A and B in the package are in the form of a capsule and tablet, respectively. Acceptable formulations of drugs A and B are tablulated below:

| | | Percentages of |
|---|---|---|
| | Acceptable Composition | Specific Strength/ |
| Formulation | of the Formulation | Dosage Form |
| Drug A 60 mg | Solid compositions containing | 14.4% fexofenadine |
| capsules | 5-180 mg of fexofenadine | hydrochloride |
| | hydrochloride in combination | 4.8% croscarmellose |
| | with: | sodium |
| | 1-10% croscarmellose | 33.8% microcrystal- |
| | sodium | line cellulose |
| | 20-85% microcrystalline | 33.8% lactose |
| | cellulose | 9.6% pregelatinized |
| | 20-85% lactose | starch |
| | 1-30% pregelatinized starch | 3.5% gelatin |
| | 1-15% gelatin | |
| Drug A tablets | Solid compositions containing | 30% fexofenadine |
| (30 mg, 60 mg, | 5-180 mg fexofenadine | hydrochloride |
| 120 mg, 180 mg) | hydrochloride in combination | 6% croscarmellose |
| | with: | sodium |
| | 1-10% croscarmellose | 33.25% microcrystal- |
| | sodium | line cellulose |
| | 20-85% microcrystalline | 30% pregelatinized |
| | cellulose | starch |
| | 5-50% pregelatinized starch | 0.75% magnesium |
| | 0.05-3% magnesium stearate | stearate |
| Drug B 60 mg/ | Fexofenadine Zone: | 17.09% fexofenadine |
| 120 mg tablet | 0.25-6.00% croscarmellose | 3.42% croscarmellose |
| | sodium | sodium |
| | 27-73% microcrystalline | 61.67% microcrystal- |
| | cellulose | line cellulose |
| | 15-30% pregelatinized starch | 17.09% pregelatinized |
| | 0.25-2.00% magnesium | starch |
| | stearate | 0.75% magnesium |
| | Pseudoephedrine Zone: | stearate |
| | 59-81% carnauba wax | 28.17% pseudo- |
| | 0.25-2% stearic acid | ephedrine |
| | 0-3% colloidal silicon | hydrochloride |
| | dioxide | 70.42% carnauba wax |
| | | 1.15% stearic acid |
| | | 0.25% colloidal silicon |
| | | dioxide |

Drug A contains about 14.4% by weight of fexofenadine hydrochloride, 5 mg to 180 mg and which has a particle surface greater than 1.0 m²/g. In one aspect of the invention, the 5 particle surface ranges from 2 m²/g to 10 m²/g. In a further preferred embodiment, the particle surface ranges from 2 m²/g to 6 m²/g. And in another preferred embodiment, the particle surface ranges from 2 m²/g to 4 m²/g. Drug A contains further ingredients, at least one of which is an inert ingredient. The acceptable inert ingredients can be selected from the group consisting of croscarmellose sodium, lactose, microcrystalline cellulose, pregelatinized starch, gelatin, calcium carbonate, magnesium stearate and sodium starch glycolate.

In one aspect of the invention, the package containing drug A in accordance with this invention contains the following inert ingredients: croscarmellose sodium, microcrystalline cellulose, lactose, pregelatinized starch and gelatin. The respective amounts of such ingredients range from 1-10%, 20%-85%, 20%-85%, 1-30%, 1-15%, by weight of drug A.

In a further preferred embodiment, the package according to this invention containing drug A contains croscarmellose sodium, microcrystalline cellulose, lactose, pregelatinized starch and gelatin in amounts of about 4.8%, 33.8%, 33.8%, 9.6%, and 3.5%, respectively, by weight of drug A.

In another aspect of the invention, the package contains drug A having the following inert ingredients: microcrystalline cellulose, pregelatinized starch, gelatin, magnesium stearate, calcium carbonate and sodium starch glycolate. The respective amounts of such ingredients range from 20-85%, 5-50%, 1-15%, 0.05-3%, 5-50% and 1-15%, by weight of drug A.

In a further preferred embodiment, the package according to this invention contains drug A, which essentially contains microcrystalline cellulose, pregelatinized starch, gelatin, magnesium stearate, calcium carbonate and sodium starch glycolate in amounts of about 33.5%, 28.3%, 3.1%, 0.5%, 15.0% and 5.4%, respectively, by weight of drug A.

In another aspect of the invention, the drug A in accordance with this invention contains the following inert ingredients: croscarmellose sodium, microcrystalline cellulose, lactose, pregelatinized starch, gelatin, and magnesium stearate. The respective amounts of such ingredients range from 1-10%, 20-85%,20-85%,1-30%,1-15% and 0.05-3%, by weight of drug A.

In a further preferred embodiment, the drug A contains croscarmellose sodium, microcrystalline cellulose, lactose, pregelatinized starch, gelatin, and magnesium stearate in amounts of about 4.6%, 32.4%, 32.4%, 9.2%, 3.4% and 0.5%, respectively, by weight of drug A.

In a further preferred embodiment, the package according to this invention contains drug A, which contains croscarmellose sodium, microcrystalline cellulose, lactose, pregelatinized starch, gelatin, and magnesium stearate in amounts of about 4.8%, 33.7%, 33.7%, 9.6%, 3.5% and 0.5%, respectively, by weight of drug A.

In another aspect of the invention, the drug A in accordance with this invention contains the following inert ingredients: microcrystalline cellulose, pregelatinized starch, magnesium stearate, calcium carbonate, and sodium starch glycolate. The respective amounts of such ingredients range from 20-85%, 5-50%, 0.05-3%, 5-50%, and 1-15% by weight of drug A.

In a further preferred embodiment, the package according to this invention contains drug A, which contains microcrystalline cellulose, pregelatinized starch, magnesium stearate, calcium carbonate, and sodium starch glycolate in amounts of about 35.1%, 29.8%, 0.5%, 15.0%, and 5.4%, respectively, by weight of drug A.

Drug B is a bilayer tablet that contains two zones. The first discrete zone contains a therapeutically effective decongestant amount of pseudoephedrine, or a pharmaceutically acceptable addition salt thereof, in an amount of about 18% to about 39% by weight of pseudoephedrine, most preferably in an amount of about 25% to 33%, and a first carrier base material, the first carrier base material comprising a mixture of; (i) carnauba wax in an amount of about 59% to about 81% by weight of pseudoephedrine, most preferably in an amount of about 66% to about 74%; and (ii) a suitable antiadherent in an amount of about 0.25% to about 2.00%, most preferably in an amount of about 0.50% to about 1.50% by weight of pseudoephedrine; wherein the first carrier base material provides a sustained release of the pseudoephedrine or a pharmaceutically acceptable addition salt thereof. The second discrete zone contains a therapeutically effective antihistaminic amount of fexofenadine, or a pharmaceutically acceptable addition salt thereof, such as fexofenadine hydrochloride of the formula;

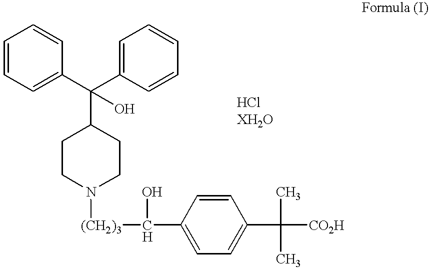

wherein X is a number ranging from about zero to about 5, and the individual optical isomers thereof, in an amount of about 15% to about 30%, most preferably in an amount of about 15% to about 24% by weight of fexofenadine and a second carrier base material, the second carrier base comprising a mixture of; (i) a cellulose diluent in an amount of about 27% to about 73%, most preferably in an amount of about 43% to about 67% by weight of fexofenadine; (ii) pregelatinized starch in an amount of about 15% to about 30%, most preferably in an amount of about 15% to about 24% by weight of fexofenadine; (iii) a suitable disintegrant in an amount of about 0.25% to about 6.00%, most preferably in an amount of about 3.20% to about 4.80% by weight of fexofenadine; and (iv) a suitable lubricant in an amount of about 0.25% to about 2.00%, most preferably in an amount of about 0.50% to about 1.00% by weight of fexofenadine. The second carrier base material provides an immediate release of fexofenadine or the pharmaceutically acceptable addition salt. The package in accordance with this invention contains pseudoephedrine as pseudoephedrine hydrochloride.

The compound name of 4-[4-[4-(hydroxydipheny1methyl)-1-piperdinyl]-1-hydroxybutyl] -α,α-dimethylbenzeneacetic acid hydrochloride is the equivalent of another chemical name, fexofenadine hydrochloride. See U.S. Pat. No. 5,855,912 which is herein incorporated by reference.

As used herein the term “fexofenadine hydrochloride or a pharmaceutically acceptable addition salt thereof” corresponds to the formula as described above wherein X is a number ranging from about zero to 5, and the individual optical isomers thereof. The compound 4-[4-[4-(hydroxydiphenylmethyl)-1-piperdinyl]-1-hydroxybutyl]-α,α-dimethylbenzeneacetic acid hydrochloride wherein X is zero or one in the formula (I) is the most preferred form of fexofenadine. The package in accordance with this invention contains pseudoephedrine as pseudoephedrine hydrochloride.

A suitable glidant, such as colloidal silicon dioxide, that is included in the first carrier base material of pseudoephedrine in an amount of 0.00% to about 3.00% by weight of pseudoephedrine and more preferred in an amount of 0.00% to about 0.75% by weight of pseudoephedrine. Stearic acid is the suitable antiadherent of pseudoephedrine, a suitable disintegrant in fexofenadine is croscarmellose sodium and the suitable lubricant is magnesium stearate. The pseudoephedrine hydrochloride, camauba wax, stearic acid and colloidal silicon dioxide of pseudoephedrine are combined in amounts of about 28.17%, about 70.42%, about 1.15% and about 0.25% respectively, by weight of the composition of pseudoephedrine, and the fexofenadine, cellulose diluent, pregelatinized starch, croscarmellose sodium and magnesium stearate of fexofenadine are combined in amounts of about 17.09%, about 61.67%, about 17.09%, about 3.42% and about 0.75% respectively, by weight of the composition of fexofenadine. The term fexofenadine refers to fexofenadine hydrochloride.

As used herein, the cellulose diluent comprises a combination of AVICEL® PH101 and AVICEL PH 102 in amounts of about 12% and 88% respectively. In addition the fexofenadine hydrochloride is present in an amount of about 60 mg and the pseudoephedrine hydrochloride is present in an amount of about 120 mg. The bi-layer tablets that are coated with a suitable coating agent such as OPADRY® YS-1-7006 and have a hardness of about 15 kp to about 25 kp. The coating agent of OPADRY® YS-1-7006 is present in amounts of about 2.9% by weight of the composition. The term “pseudoephedrine hydrochloride” (See U.S. Pat. No. 6,039,974 herein incorporated by reference) or a “pharmaceutically acceptable addition salt thereof” corresponds to the formula;

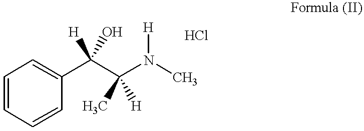

The term “pharmaceutically acceptable salt” refers to those salts of formulas (I) and (II) that are not substantially toxic at the dosage administered to achieve the desired effect and do not independently possess significant pharmacological activity. The salts included within the scope of this term are pharmaceutically acceptable acid addition salts of a suitable inorganic or organic acid. Suitable inorganic acids are, for example hydrochloric, hydrobromic, sulfuric and phosphoric acids. Suitable organic acids include carboxylic acids, such as acetic, propionic, glycolic, lactic, pyruvic, malonic, succinic, fumaric, malic, tartaric, citric, cyclamic, ascorbic, maleic, hydroxymaleic, dihydroxymaleic, benzoic, phenylacetic, 4-aminobenzoic, 4hydroxybenzoic, anthranillic, cinnamic, salicylic, 4-aminosalicyclic, 2-phenoxybenzoic, 2-acetoxybenzoic and mandelic acid, sulfonic acids, such as methanesulfonic, ethanesulfonic and β-hydroxyethanesulfonic acid. In addition, pharmaceutically acceptable salts include those salts of formulas (I) and (II) formed with inorganic and organic bases, such as those of alkali metals, for example sodium, potassium and lithium, alkaline earth metals, for example calcium and magnesium, light metals of group IIIA, for example aluminum, organic amines, for example primary, secondary or tertiary amines, such as cyclohexylamine, ethylamine, pyridine, methylaminoethanol and piperazine. The salts are prepared by conventional means known by one of ordinary skill in the art as, for example, by treating a compound of formulas (I) and (II) with an appropriate acid or base. Such salts can exist in either a hydrated or substantially anhydrous form. The preferred acid addition salts are those prepared from hydrochloric acid, sulfuric acid and tartaric acid.

The term “stereoisomers” is a general term for all isomers of individual molecules that differ only in the orientation of their atoms in space. It includes geometric (cis/trans) isomers, and isomers of compounds with more than one chiral center that are not mirror images of one another (diastereomers).

As used herein, the term “cellulose diluent” includes microcrystalline cellulose, AVICEL PH101, AVICEL PH102, AVICEL PH301, AVICEL PH302, AVICEL PH200, AVICEL PH112, AVICEL PH113, AVICAL PH103, AVICAL PH105 and the like. The preferred cellulose diluent is microcrystalline cellulose, AVICEL PH101 and AVICEL PH102, and the most preferred cellulose diluent is a combination of AVICEL PH101 and AVICEL PH102. It is especially preferred that the AVICEL PH101 and AVICEL PH102 mixture comprise about 12% AVICEL PH101 and about 88% AVICEL PH102.

As used herein, the term “suitable antiadherent” includes stearic acid, cetyl alcohol, stearyl alcohol, paraffin, white wax, glycerin, lanolin, talc, mineral oil and the like. The preferred suitable antiadherent is stearic acid.

As used herein, the term “suitable disintegrant” includes croscarmellose sodium, crospovidone, alginic acid, sodium alginate, methacrylic acid DVB, cross-linked PVP, microcrystalline cellulose, polacrilin potassium, sodium starch glycolate, starch, pregelatinized starch and the like. The preferred suitable disintegrant is croscarmellose sodium.

As used herein, the term “suitable lubricant” includes magnesium stearate, calcium stearate, zinc stearate, stearic acid, talc, hydrogenated vegetable oil and the like. The preferred suitable lubricant is magnesium stearate.

As used herein, the term “suitable glidant” includes silicon dioxide, talc and the like. The preferred suitable glidant is silicon dioxide.

As used herein the term “inert ingredient” refers to those therapeutically inert ingredients that are well known in the art of pharmaceutical science which can be used singly or in various combinations, and include, for example, binders, diluents, lubricants, glidants, sweetening agents, disintegrants, coloring agents, flavoring agents, antioxidants, solubilizing agents, coating agents and the like, as are disclosed in The United States Pharmacopeia, XXII, 1990, (1989 The United States Pharmacopeial Convention, Inc.), pages 1857-1859, which is incorporated herein by reference. For example, the following inert ingredients can be utilized singly or in various combinations; binders such as gelatin, polyvinylpyrrolidone (PVP), pregelatinized starch, povidone; diluents such as calcium carbonate, lactose, starch, microcrystalline cellulose, and the like; lubricants such as magnesium stearate, calcium stearate, zinc stearate, stearic acid, talc, hydrogenated vegetable oil and the like; glidants such as silicon dioxide, talc and the like; disintegrants such as alginic acid, methacrylic acid DVB, cross-linked PVP, microcrystalline cellulose, croscarmellose sodium, crospovidone, polacrilin potassium, sodium starch glycolate, starch, pregelatinized starch and the like; sweetening agents; coloring agents; flavoring agents; antioxidants; and the like.

### DESCRIPTION OF THE PREFERRED EMBODIMENT

One embodiment of the present invention will now be described with reference to the accompanying drawings wherein:

Referring to FIG. I of the drawings, the package is in the form of a rectangular box shown as the cut out frontal view of the interior of the box 3; comprising bottles 1 and 2 that contains Drug A and B with lids 1a and 2a respectively; bottle 1 resting on the platform 7; with the top cover flap 5 of the box in a closed position and the top side flaps 6 in a closed position; 4 as the side view of the box and 8 as the top view of the box.

Referring to FIG. II of the drawings, the package is in the form of a rectangular box showing the uncut frontal view 3′ of the box with the top cover flap 5′ open and the top side flaps 6′ open; affixed to the top side flaps 6′ is a cushion 6a′ and 4 as the side view of the box.

Referring to FIG. III of the drawings, the container is shown as an open view of bottles 1 or 2; with the lid of the bottle 1a or 2a positioned directly above the bottle.

1. A package to dispense two or more pharmaceutically active compounds comprising:
(a) a container 1 to dispense a drug A having therapeutically effective amounts of fexofenadine or a pharmaceutically acceptable addition salt thereof;
(b) a container 2 to dispense a drug B having therapeutically effective amounts of fexofenadine and pseudoephedrine or their pharmaceutically acceptable addition salts; and
wherein there is provided an indicia to distinguish the drugs A and B in the containers 1 and 2.

2. The package of claim 1 wherein the addition salt is fexofendadine hydrochloride having the formula 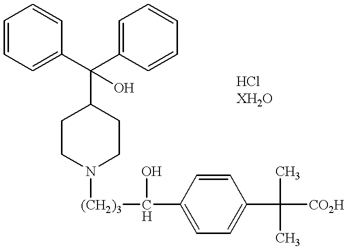
wherein x is a number ranging from about zero to 5, and the individual optical isomers thereof.

3. The package of claim 1 wherein the addition salt is pseudoephedrine hydrochloride having the formula

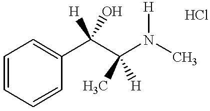

4. The package according to claim 1, wherein drug A of the container 1 is indicated for nighttime use and drug B in the container 2 is indicated for daytime use.

5. The package according to claim 4, wherein an indicia is used for distinguishing between container 1 and container 2.

6. The package according to claim 5 wherein the indicia is size or color.

7. The package according to claim 6, wherein drug A is in a smaller container.

8. The package according to claim 6, wherein drug B is in a larger container.

9. The package according to claim 1, wherein container 1 and container 2 are in the form of medicinal bottles.

10. The package according to claim 1, wherein containers 1 and 2 are enclosed in a uni-package.

11. The uni-package according to claim 10 wherein the package is a convenient package.

12. The uni-package according to claim 11 which is in the form of a box.

13. The package according to claim 1, wherein drugs A and B in the containers 1 and 2 are in the form of capsules, caplets, tablets or liquid formulations.

14. The container according to claim 13, wherein the drug A in container 1 is a capsule.

15. The container according to claim 13, wherein the drug B in container 2 is a tablet.

16. The package according to claim 1, wherein the drug A and the drug B further comprise a pharmaceutical carrier and a formulating aid.

17. The package according to claim 16, wherein the drug A contains fexofenadine hydrochloride and has a particle surface area of greater than about 1.0 m²/g; and
additionally contains at least one inert ingredient.

18. The package according to claim 17, wherein at least one inert ingredient is selected from the group consisting of croscarmellose sodium, lactose, microcrystalline cellulose, pregelatinized starch, gelatin, calcium carbonate, magnesium stearate and sodium starch glycolate.

19. The package according to claim 18 wherein the inert ingredients comprise croscarmellose sodium, lactose, microcrystalline cellulose, pregelatinized starch and gelatin.

20. The package according to claim 19 wherein the croscarmellose sodium, the microcrystalline cellulose, the lactose, the pregelatinized starch and the gelatin are present in amounts of about 1% to about 10%, 20% to about 85%, 20% to about 85%, 1% to about 30% and 1% to about 15%, respectively, by weight of drug A.

21. The package according to claim 19 wherein the croscarmellose sodium, the microcrystalline cellulose, the lactose, the pregelatinized starch and the gelatin are present in amounts of about 4.8%, 33.8%, 33.8%, 9.6% and 3.5%, respectively, by weight of drug A.

22. The package according to claim 18 wherein the inert ingredients comprise microcrystalline cellulose, pregelatinized starch, gelatin, magnesium stearate, calcium carbonate and sodium starch glycolate.

23. The package according to claim 22 wherein the microcrystalline cellulose, the pregelatinzied starch, the gelatin, the magnesium stearate, the calcium carbonate and the sodium starch glycolate are present in amounts of about 20% to about 85%, 5% to about 50%, 1% to about 15%, 0.05% to about 3%, 5% to about 50% and 1% to about 15%, respectively, by weight of drug A.

24. The package according to claim 22 wherein the microcrystalline cellulose, the pregelatinized starch, the gelatin, the magnesium stearate, the calcium carbonate and the sodium starch glycolate are present in amounts of about 33.5%, 28.3%, 3.1%, 0.5%,15.0%, 5.4%, respectively, by weight of drug A.

25. The package according to claim 18 wherein the inert ingredients comprise croscarmellose sodium, microcrystalline cellulose, lactose, pregelatinized starch, gelatin and magnesium stearate.

26. The package according to claim 25 wherein the croscannellose sodium, the microcrystalline cellulose, the lactose, the pregelatinized starch, the gelatin and the magnesium stearate are present in amounts of about 1% to about 10%, 20% to about 85%, 20% to about 85%, 1% to about 30%, 1% to about 15% and 0.05% to about 3.0%, respectively, by weight of drug A.

27. The package according to claim 25 wherein the croscarmellose sodium, the microcrystalline cellulose, the lactose, the pregelatinized starch, the gelatin and the magnesium stearate are present in amounts of about 4.6%, 32.4%, 32.4%, 9.2%, 3.4% and 0.5%, respectively, by weight of drug A.

28. The package according to claim 25 wherein the croscarmellose sodium, the microcrystalline cellulose, the lactose, the pregelatinized starch, the gelatin and the magnesium stearate are present in amounts of about 4.8%, 33.7%, 33.7%, 9.6%, 3.5% and 0.5%, respectively, by weight of drug A.

29. The package according to claim 18 wherein the inert ingredients comprise microcrystalline cellulose, pregelatinized starch, magnesium stearate, calcium carbonate and sodium starch glycolate.

30. The package according to claim 29 wherein the microcrystalline cellulose, the pregelatinized starch, the magnesium stearate, the calcium carbonate and the sodium starch glycolate are present in amounts of about 20% to about 85%, 5% to about 50%, 0.05% to about 3%, 5% to about 50% and 1% to about 15%, respectively, by weight of drug A.

31. The package according to claim 29 wherein the microcrystalline cellulose, the pregelatinized starch, the magnesium stearate, the calcium carbonate and the sodium starch glycolate are present in amounts of about 35.1%, 29.8%, 0.5%, 15.0%, and 5.4%, respectively, by weight of drug A.

32. The package according to claim 18 wherein the drug A is fexofenadine hydrochloride.

33. The package according to claim 32 wherein fexofenadine hydrochloride is present in an amount of about 14.4% by weight of drug A.

34. The package according to claim 32 wherein fexofenadine hydrochloride has a particle surface area of about 2 m²/g to about 10 m²/g.

35. The package according to claim 32 wherein fexofenadine hydrochloride has a particle surface area of about 2 m²/g to about 6 m²/g.

36. The package according to claim 32 wherein fexofenadine hydrochloride has a particle surface area of about 2 m²/g to about 4 m²/g.

37. The package according to claim 32 wherein fexofenadine hydrochloride is present in an amount of about 5 mg to about 180 mg.

38. The package according to claim 16 wherein drug B is a bilayer tablet comprising,
(a) a first discrete zone containing a therapeutically effective decongestant amount of pseudoephedrine, or a pharmaceutically acceptable addition salt thereof, in an amount of about 18% to about 39% by weight of pseudoephedrine, and a first carrier base material, the first carrier base material comprising a mixture of;
(i) camauba wax in an amount of about 59% to about 81% by weight of pseudoephedrine; and
(ii) a suitable antiadherent in an amount of about 0.25% to about 2.00% by weight of pseudoephedrine;
wherein the first carrier base material provides a sustained release of the pseudoephedrine or a pharmaceutically acceptable addition salt thereof; and
(b) a second discrete zone containing a therapeutically effective antihistaminic amount of fexofenadine, or a pharmaceutically acceptable addition salt thereof, in an amount of about 15% to about 30% by weight of fexofenadine and a second carrier base material, the second carrier base comprising a mixture of;
(i) a cellulose diluent in an amount of about 27% to about 73% by weight of fexofenadine;
(ii) pregelatinized starch in an amount of about 15% to about 30% by weight of fexofenadine;
(iii) a suitable disintegrant in an amount of about 0.25% to about 6.00% by weight of fexofenadine; and
(iv) a suitable lubricant in an amount of about 0.25% to about 2.00% by weight of fexofenadine;
wherein the second carrier base material provides an immediate release of fexofenadine or the pharmaceutically acceptable addition salt thereof.

39. The package according to claim 38 wherein the addition salt is fexofenadine hydrochloride having the formula; 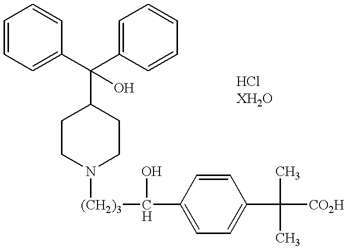
wherein X is a number ranging from about zero to about 5, and the individual optical isomers thereof.

40. The package according to claim 38 wherein the bi-layer tablet of drug B comprises,
(a) a first discrete zone containing a therapeutically effective decongestant amount of a pseudoephedrine, or a pharmaceutically acceptable addition salt thereof in an amount of about 25% to about 33% by weight of pseudoephedrine, and a first carrier base material, the first carrier base material comprising a mixture of;
(i) camauba wax in an amount of about 66% to about 74% by weight of pseudoephedrine; and
(ii) a suitable antiadherent in an amount of about 0.50% to about 1.50% by weight of pseudoephedrine;
wherein the first carrier base material provides a sustained release of the pseudoephedrine or a pharmaceutically acceptable addition salt thereof; and
(b) a second discrete zone made with fexofenadine which comprises a therapeutically effective antihistaminic amount of fexofenadine hydrochloride of the formula; 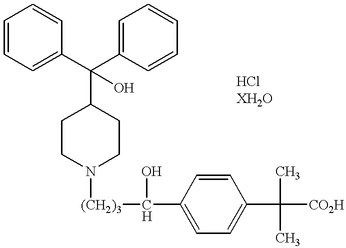
wherein X is a number ranging from about zero to about 5, and the individual optical isomers thereof, in an amount of about 15% to about 24% by weight of fexofenadine and a second carrier base material, the second carrier base comprising a mixture of;
(i) a cellulose diluent in an amount of about 43% to about 67% by weight of fexofenadine;
(ii) pregelatinized starch in an amount of about 15% to about 24% by weight of fexofenadine;
(iii) a suitable disintegrant in an amount of about 3.20% to about 4.80% by weight of fexofenadine; and
(iv) a suitable lubricant in an amount of about 0.50% to about 1.00% by weight of fexofenadine;
wherein the second carrier base material provides an immediate release of the pseudoephedrine or the pharmaceutically acceptable addition salt thereof.

41. The package according to claim 38 wherein a suitable glidant is included in the first carrier base material of pseudoephedrine in an amount of 0.00% to about 3.00% by weight of pseudoephedrine.

42. The package according to claim 41 wherein a suitable glidant is included in the first carrier base material of pseudoephedrine in an amount of 0.00% to about 0.75% by weight of pseudoephedrine.

43. The package according to claim 42 wherein the suitable glidant is colloidal silicon dioxide.

44. The package according to claim 43 wherein the pseudoephedrine is pseudoephedrine hydrochloride.

45. The package according to claim 40 wherein the pseudoephedrine is pseudoephedrine hydrochloride.

46. The package according to claim 44 wherein the suitable antiadherent of pseudoephedrine is stearic acid, and in fexofenadine, the suitable disintegrant is croscarmellose sodium and the suitable lubricant is magnesium stearate.

47. The package according to claim 45 wherein the suitable antiadherent of pseudoephedrine is stearic acid, and in fexofenadine, the suitable disintegrant is croscarmellose sodium and the suitable lubricant is magnesium stearate.

48. The package according to claim 46 wherein the pseudoephedrine hydrochloride, camauba wax, stearic acid and colloidal silicon dioxide of pseudoephedrine are combined in amounts of about 28.17%, about 70.42%, about 1.15% and about 0.25% respectively, by weight of the composition of pseudoephedrine, and the fexofenadine, cellulose diluent, pregelatinized starch, croscarmellose sodium and magnesium stearate of fexofenadine are combined in amounts of about 17.09%, about 61.67%, about 17.09%, about 3.42% and about 0.75% respectively, by weight of the composition of fexofenadine.

49. The package according to claim 47 wherein the pseudoephedrine hydrochloride, camauba wax, stearic acid and colloidal silicon dioxide of pseudoephedrine are combined in amounts of about 28.17%, about 70.42%, about 1.15% and about 0.25% respectively, by weight of the composition of pseudoephedrine, and the fexofenadine, cellulose diluent, pregelatinized starch, croscarmellose sodium and magnesium stearate of fexofenadine are combined in amounts of about 17.09%, about 61.67%, about 17.09%, about 3.42% and about 0.75% respectively, by weight of the composition of fexofenadine.

50. The package according to claim 48 wherein the fexofenadine is fexofenadine hydrochloride.

51. The package according to claim 49 wherein the fexofenadine is fexofenadine hydrochloride.

52. The package according to claim 50 wherein the cellulose diluent comprises a combination of AVICEL PH101 and AVICEL PH102.

53. The package according to claim 51 wherein the cellulose diluent comprises a combination of AVICEL PH101 and AVICEL PH102.

54. The package according to claim 52 wherein the combination of AVICEL PH101 and AVICEL PH102 comprises about 12% AVICEL PH101 and about 88% AVICEL PH102.

55. The package according to claim 53 wherein the combination of AVICEL PH101 and AVICEL PH102 comprises about 12% AVICEL PH101 and about 88% AVICEL PH102.

56. The package according to claim 54 wherein the fexofenadine hydrochloride is present in an amount of about 60 mg and the pseudoephedrine hydrochloride is present in an amount of about 120 mg.

57. The package according to claim 55 wherein the fexofenadine hydrochloride is present in an amount of about 60 mg and the pseudoephedrine hydrochloride is present in an amount of about 120 mg.

58. The package according to claim 56 wherein the bilayer tablet is coated with a suitable coating agent.

59. The package according to claim 57 wherein the bilayer tablet is coated with a suitable coating agent.

60. The package according to claim 58 wherein the bilayer tablet is coated with OPADRYR® YS-1-7006.

61. The package according to claim 59 wherein the bilayer tablet is coated with OPADRY® YS-1-7006.

62. The package according to claim 60 wherein the OPADRYO® YS-1-7006 is present in amount of about 2.9% by weight of the composition.

63. The package according to claim 61 wherein the OPADRY® YS-1-7006 is present in amount of about 2.9% by weight of the composition.

64. The package according to claim 56 wherein the bilayer tablet has a hardness of about 15 kptoabout 25 kp.

65. The package according to claim 57 wherein the bilayer tablet has a hardness of about 15 kp to about 25 kp.
